(19) United States
(12) Patent Application Publication
(10) Pub. No.: US 2026/0232695 A1
(43) Pub. Date: Aug. 13, 2026

(54) ORAL FORMULATIONS OF ABIRATERONE ACETATE AND METHOD OF MANUFACTURING THEREOF

(71) Applicant: BDR PHARMACEUTICALS INTERNATIONAL PRIVATE LIMITED, Mumbai, Maharashtra (IN)

(72) Inventors: Dharmesh Mahendrabhai SHAH, Mumbai, Maharashtra (IN); Aravind Manappa BADIGER, Halol, Gujarat (IN); Mukeshkumar Subhashchandra SHARMA, Halol, Gujarat (IN); Madhavkumar Dilipbhai TRIVEDI, Halol, Gujarat (IN); Samirkumar Babulal PANCHAL, Halol, Gujarat (IN); Nilay Manikant JAYASWAL, Halol, Gujarat (IN); Akshaykumar Sampatsinh JADAV, Halol, Gujarat (IN)

(21) Appl. No.: 19/150,679

(22) PCT Filed: Aug. 5, 2023
(86) PCT No.: PCT/IN2023/050753

§ 371 Date: Jul. 24, 2025

(30) Foreign Application Priority Data
Aug. 5, 2022 (IN) 202221044843

(51) Int. Cl. A61K 31/58; A61K 9/10; A61K 47/02; A61K 47/10; A61K 47/14; A61K 47/26; A61K 47/32; A61K 47/34; A61K 47/36; A61K 47/38; A61K 47/44

(52) U.S. Cl. CPC A61K 31/58; A61K 9/10; A61K 47/02; A61K 47/10; A61K 47/14; A61K 47/26; A61K 47/32; A61K 47/34; A61K 47/36; A61K 47/38; A61K 47/44

(57) ABSTRACT

The present invention relates to oral suspension dosage form of Abiraterone Acetate. The invention also relates to provide patient-compliant, economical and technically advanced dosage form over existing dosage form. Moreover, the solubility and stability of the patient compliant Abiraterone Acetate formulation, prepared as per the present invention, is proven higher when compared to prior art inventions. Furthermore, the present invention also provides a suspension composition prepared by a process which is relatively simple, easy to commercially manufacture, and functionally reproducible.

### RELATED APPLICATIONS

The present application is the National Stage entry of International Patent Application No. PCT/IN2023/050753, filed on Aug. 5, 2023, which is based on and claims priority to Indian Patent Application No. 202221044843, filed on Aug. 5, 2022, which are incorporated herein by reference in their entirety.

### FIELD OF THE INVENTION

The present invention relates to oral liquid formulation of Abiraterone Acetate as a suspension. Further, the present invention relates to providing an economical and technically advanced dosage form over existing dosage form.

### BACKGROUND OF THE INVENTION

Abiraterone Acetate (CAS: 154229-18-2) is an inhibitor of CYP17 (17α-hydroxylase/C17,20-lyase). Abiraterone Acetate is chemically known as 17-(pyridin-3-yl)androsta-5,16-dien-3β-yl acetate and is represented structurally as below:

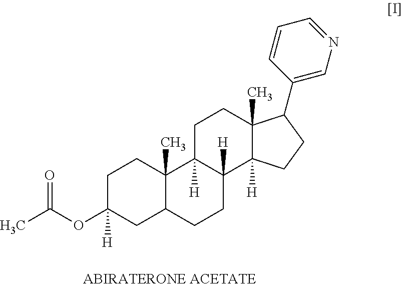

WO1993/20097 first discloses Abiraterone Acetate and analogues (Markush structure) and their use in treating of prostatic cancer. WO2006/021776 further discloses methanesulfonic acid salt of Abiraterone Acetate.

Abiraterone Acetate is marketed presently under the brand name “ZYTIGA®” in United States. This tablet is indicated in combination with prednisone for the treatment of patients with metastatic castration-resistant prostate cancer (CRPC) & metastatic high-risk castration-sensitive prostate cancer (CSPC). The recommended dosage for Abiraterone Acetate is 1,000 mg (two 500 mg tablets or four 250 mg tablets) orally once daily with prednisone 5 mg orally twice daily.

U.S. Pat. No. 8,822,438 claims a method for the treatment of a prostate cancer in a human comprising administering to said human a therapeutically effective amount of Abiraterone Acetate or a pharmaceutically acceptable salt thereof and a therapeutically effective amount of prednisone.

CN102743393 discloses tablet compositions having the same qualitative composition preparation method thereof and the same concentration of Abiraterone Acetate in the tablet as in the Zytiga tablet.

CN102336801 discloses a tablet comprising a high load of Abiraterone Acetate, however the dissolution profile of this composition is slower when comparing to the approved Zytiga 250 mg tablet.

WO2013/164473 discloses Abiraterone Acetate dissolved or dispersed in a carrier, wherein the carrier comprises one or more lipid excipients.

U.S. Pat. No. 9,511,078 claims pharmaceutical formulation comprising Abiraterone, propylene glycol monocaprylate and an emulsifier. It discloses pharmaceutical formulation comprising low aqueous solubility drug, such as Abiraterone formulated in a capsule, wherein the drug is solubilized within the other components of the pharmaceutical formulation which forms a nanoemulsion upon exposure to an aqueous environment.

WO2014/009436 discloses Nanosuspension comprising particles of Abiraterone Acetate or a pharmaceutically acceptable salt, hydrate or solvate thereof having a d(0.5) of less than 1000 nm.

WO2014/145813 discloses method for producing a composition comprising nanoparticles of Abiraterone Acetate, the method comprising dry milling a composition comprising Abiraterone Acetate, a millable grinding compound, a facilitating agent and one or both of an antioxidant and a sequestering agent in a mill comprising a plurality of milling bodies, for a time period sufficient to produce a composition comprising fine particles of the Abiraterone Acetate, wherein the particle size of the grinding matrix and the particle size of the Abiraterone Acetate is reduced by dry milling.

WO2015/032873 discloses a tablet comprising a high load of Abiraterone Acetate of 50 to 80% w/w and one wetting agent. However, when comparing to the approved Zytiga 250 mg tablet the bioavailability of these composition is low.

WO2015/114314 discloses a dosage form composition of Abiraterone Acetate in the form of an emulsion, solution, suspension, syrup or elixir for once daily administration wherein the Abiraterone particles are nano-sized with a particle size of less than about 2000 nanometers.

U.S. Ser. No. 10/722,527 discloses lipid compositions for Abiraterone Acetate, such as lipid multiparticulate formulations comprising Abiraterone Acetate and a lipid matrix or liquid fill hard capsules comprising Abiraterone Acetate and a lipid matrix. However, the said invention is not considered as friendly because capsules are subject to the effects of relative humidity and microbial contamination.

WO2017/037647 discloses an oral pharmaceutical formulation comprising Abiraterone, solubilizing agents and other pharmaceutically acceptable excipients, wherein the formulation comprises less than 1000 mg of Abiraterone.

WO2019/186444 discloses oral emulsion formulation of Abiraterone comprising Abiraterone, polysorbate-80, lecithin an oil phase and an aqueous phase.

WO2019/206472 discloses a tablet composition for oral administration of Abiraterone Acetate, particularly to pharmaceutical granulates and tablets giving immediate release of Abiraterone Acetate in the stomach.

Most of the prior-art is of oral tablet formulation of Abiraterone Acetate. These tablets are large and required to be taken on empty stomach. Hence, to overcome the problems of the prior art, there is still need of finding an oral liquid dosage form of Abiraterone, which is also bioequivalent to the commercial Abiraterone tablet Zytiga®.

Abiraterone Acetate is a challenging process due to its less soluble and less permeable (BCS Class-IV product) nature and it may be noted suspensions are a useful drug delivery system for therapeutic agents that have a low solubility. Therefore, drugs with poor wetting and slow dissolution properties may be difficult to formulate and manufacture as a tablet that will provide adequate of full drug bioavailability and suspension will enhance dissolution and bioavailability.

In addition, tables are not suitable for patients with dysphagia and old age patients because they cannot take it easily and some drugs cause gastric irritation when they are given in the tablet form. Further, tablet is not suitable for unconscious patients. Above limitations were identified quite effectively by the inventors of the present invention and hence, a stable-efficacious oral liquid suspension dosage form has been developed to meet the identified shortfalls in the prior-art publications.

The present invention discloses Abiraterone Acetate in an oral liquid suspension dosage form that does not use sophisticated techniques and is economically affordable compared to available dosage form. Further, the formulated oral liquid suspension dosage form prepared as per the present invention provides greater stability. In addition, oral liquid dosage forms are the most suitable dosage form for patients who have difficulty taking tablets or capsules, as might be the case with patients with dysphagia, pediatric or geriatric patients. They are attractive in appearance and gives beneficial psychological effects.

A suspension is a heterogeneous mixture that contains solid particles sufficiently large for sedimentation. The suspended particles may be visible to the naked eye. A suspension is a heterogeneous mixture in which the suspended particles do not dissolve, although they remain suspended throughout the bulk of the liquid medium. Generally, the solid part is dispersed through mechanical agitation using suspending agents. In contrast, solution have dissolved and homogeneously mixed solute which does not exist as a solid.

The present invention incorporates smaller particle size of suspension that increases the solubility of Abiraterone Acetate by providing better dissolution profile. Moreover, the present invention resulted in achieving higher stability of Abiraterone Acetate. Moreover, the solubility and stability of the patient compliant Abiraterone Acetate formulation, prepared as per the present invention, is proven higher when compared to prior art inventions.

Further, the present invention provides a suspension composition prepared by a process which is relatively simple, easy to commercially manufacture, and functionally reproducible. Additionally, a suspension composition of the present invention is also able to incorporate two or more active ingredients.

### OBJECTIVE OF THE INVENTION

The prime objective of the present invention is to provide an oral composition of Abiraterone Acetate or a pharmaceutically acceptable salt thereof preferably as liquid suspension dosage form with one or more pharmaceutically acceptable excipients and method of preparation thereof.

In one objective of the present invention the formulated product is a stabilized suspension.

In one objective of the present invention, wherein the active ingredient incorporated in the pharmaceutical composition comprises Do of particle size in the range of 0.5 to 50 microns.

Another objective according to the present invention, wherein the formulated product has particle size ranging from nanometer to micrometer, which results in enhanced in-vitro dissolution profile about 80% to 110%.

One objective of the present invention includes a pharmaceutical composition comprising Abiraterone Acetate with pharmaceutically acceptable excipients but devoid of pH adjusting agents.

In yet another objective, the pharmaceutical composition manufactured as per the present invention is a suspension. The pharmaceutical composition manufactured by number of stages in manufacturing process including homogenization, sonication, mixing and/or evaporation by spray drying.

One objective of the present invention may include a pharmaceutical composition comprising about 5% to 60% W/V of Abiraterone Acetate with pharmaceutically acceptable excipients. Wherein preferably, 5% to 45% of Abiraterone Acetate with pharmaceutically acceptable excipients and more preferably 5% to 30% of Abiraterone Acetate with pharmaceutically acceptable excipients.

In yet another objective of the present invention, wherein pH of the pharmaceutical composition is in the range of 3.5-6.5.

Another objective of the present invention is intended to be supra-bioavailable, when compared to existing tablet dosages of 2 tablets of 500 mg and 4 tablets of 250 mg against a 1000 mg/5 mL oral liquid suspension dosage form. Hence, the therapeutic dose can be reduced according to the present invention.

In yet another objective of the present invention is that composition of Abiraterone Acetate comprising solubility enhancer and permeability enhancer, which are not the part of the current dosage form available in market.

Another objective of the present invention is that the liquid formulation is administered orally once a day, while in existing dosage form is administered as four Abiraterone tablets.

Embodiments of the pharmaceutical composition may include Abiraterone Acetate as an active ingredient with one or more pharmaceutically acceptable excipients are selected from like diluent vehicles, stabilizers/anti-oxidants, suspending/thickening agents, chelating/complexing agents, solubility enhancing agents, permeability enhancers, preservatives, glidants, active carriers, sweeteners, anti-caking agents, wetting agents, preservatives, buffering agents, flavoring agents and the like.

### SUMMARY OF THE INVENTION

One aspect of the present invention relates to process for preparation of an oral liquid pharmaceutical composition as per the present invention comprising following steps:
(a) adding diluent vehicle in purified water;
(b) adding co-solvent and homogenization;
(c) adding solubilizer and homogenization;
(d) adding Abiraterone Acetate and homogenization to get homogeneous suspension;
(e) adding sweetening agent in above-prepared suspension and mixing the same through vortex or high-speed homogenizer or mechanical or sonicated or shear stress to obtain a uniform suspended formulated dosage form;
(f) adding flavouring agent and homogenization;
(g) dissolving suspending agent and thickening agent in purified water;
(h) adding solution of Step (g) into the Step (h) to provide appropriate viscosity, making-up volume of the suspension with aqueous vehicle.

The details of one or more embodiments of the invention are set forth in the description below. Other features of the invention will be apparent from the description.

### DETAILED DESCRIPTION OF THE INVENTION

The present invention will now be disclosed by describing certain preferred and optional embodiments, to facilitate various aspects thereof.

Abiraterone Acetate (CAS: 154229-18-2), chemically known as, 17-(pyridin-3-yl)androsta-5,16-dien-3β-yl acetate, its molecular weight is 391.55 g/mol. Abiraterone Acetate is considered a Bio-pharmaceutics Classification System (BCS) Class IV drug substance due to its poor solubility in water and lower lipid permeability.

In accordance with the present invention, a pharmaceutical composition comprising Abiraterone Acetate as an active ingredient with pharmaceutically acceptable excipients.

The term “pharmaceutically acceptable excipients” as used herein, refers to excipients those are routinely used in pharmaceutical compositions. The pharmaceutically acceptable excipients may comprise of diluent vehicles, stabilizers/anti-oxidants, suspending/thickening agents, chelating/complexing agents, solubility enhancing agents, preservatives, glidants, sweeteners, anti-caking agents, wetting agents, preservatives, buffering agents, flavoring agents and combinations thereof. The list of excipients used are listed in tables below although it is not limited to the said excipients.

Suitable diluent vehicle may include one or more from aqueous vehicle, sugar, methylcellulose gel, citric acid, sucrose, sorbitol solution, sodium carboxy methylcellulose solution, xanthan gum solution, non-aqueous vehicle like refined fractionated coconut oil, hydrogenated castor oil, lecithin, aluminum stearate and the like.

Suitable solubilizers may include one or more from Tween 80, Soluplus, Polyoxyl 40 Hydrogenated Castor Oil (Kolliphor® RH 40), Caprylocaproyl Polyoxyl-8 glycerides (Labrasol ALF), Lauroyl polyoxyl-6 glycerides (Labrafil M 2130 CS), Lauroyl Polyoxyl-32 glycerides (Gelucire 44/14), Cremophor, Lecithin, and the like.

Suitable suspending or thickening agents may include one or more from sodium alginate, methylcellulose, hydroxyethyl cellulose, hydroxypropyl cellulose, hydroxypropyl methylcellulose, povidone K 120, colloidal silicon dioxide, carboxymethyl cellulose, sodium carboxymethyl cellulose, acacia, tragacanth, xanthan gum, carbomer, carrageen, gelatin and the like.

Suitable co-solvent may include one or more from ethanol, propylene glycol, glycerine, glycofural, polyethylene glycols and the like.

Suitable wetting agents may include one or more from the group comprising anionic, cationic, nonionic, or zwitterionic surfactants, or combinations thereof. Suitable examples of wetting agents are sodium lauryl sulphate, cetrimide, polyethylene glycols, polyoxyethylene-polyoxypropylene block copolymers such as poloxamers, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyethylene glycol fatty acid esters such as polyoxyethylene monostearate, polyoxyethylene alkyl ethers such as polyoxyethylene lauryl ether, polyoxyethylene castor oil and the like.

Suitable permeability enhancers may include one or more from the group comprising alcohols, Polyols, short chain glycerides, amines, amides, cyclodextrins, fatty acids, pyrrolidines, azones, sulfoxides, surfactants, terpenes and the like.

Suitable surfactants or co-surfactants may include one or more from anionic, cationic, non-ionic or amphoteric surfactants. Non-limiting examples of surfactants may include polyoxyethylene-polyoxypropylene co-polymers and block co-polymers, ethoxylated cholesterins, vitamin derivatives, e.g. vitamin E derivatives such as tocopherol polyethylene glycol succinate (TPGS), sodium dodecylsulfate or sodium lauryl sulfate; a bile acid or salt thereof, for example cholic acid, glycolic acid or a salt; and the like.

Suitable sweetener may include but are not limited to one or more from sucralose, sachharin, neotame, Asparmate, Cyclamte, Glycyrrhizin, sucrose, molases, glucose, fructose, mannitol, sorbitol, xylitol, erythritol, Isomalt, maltiol, lactiol, hydrogenated starch and the like.

Suitable flavoring agents may include one or more from the group consisting of peppermint, grapefruit, orange, lime, lemon, mandarin, pineapple, strawberry, raspberry, mango, passion fruit, kiwi, apple, pear, peach, apricot, cherry, grape, banana, cranberry, blueberry, black currant, red currant, gooseberry, lingon berries, cumin, thyme, basil, camille, valerian, fennel, parsley, chamomile, tarragon, lavender, dill, bargamot, salvia, aloe vera balsam, spearmint, piperine, eucalyptus, and the like.

Suitable solubility enhancing agents may include one or more from the group comprising surfactants such as (1) non-ionic e.g., polyoxyethylene sorbitan fatty acid esters, sorbitan esters, polyoxyethylene ethers, (2) anionic e.g., sodium lauryl sulfate, sodium laurate, dialkyl sodium sulfosuccinates, particularly bis-(2-ethylhexyl) sodium sulfosuccinate, sodium stearate, potassium stearate, and sodium oleate, (3) cationic e.g., benzalkonium chloride, Cetylpyridinium chloride and Benzethonium chloride, and (4) zwitterionic/amphoteric surfactants; fatty alcohols such as lauryl, cetyl, and stearyl alcohols; glyceryl esters such as the naturally occurring mono-, di-, and tri-glycerides; fatty acid esters of fatty alcohols and other alcohols such as propylene glycol, polyethylene glycol; sucrose; polymers e.g., poloxamers, polyvinylpyrrolidones, glycerides e.g., triacetin, glyceryl monocaprylate, glyceryl monooleate, glyceryl monostearate; diethylene glycol monoethyl ether; and the like.

One embodiment of the present invention may include a pharmaceutical composition comprising about 5% to 60% W/V of Abiraterone Acetate with pharmaceutically acceptable excipients, preferably, 5% to 45% of Abiraterone Acetate with pharmaceutically acceptable excipients and more preferably 5% to 30% of Abiraterone Acetate with pharmaceutically acceptable excipients.

Another embodiment of the present invention includes a pharmaceutical composition comprising Abiraterone Acetate with pharmaceutically acceptable excipients devoid of pH adjusting agents.

In one embodiment of the present invention, wherein the active ingredient incorporated in the pharmaceutical composition comprises Do of particle size in the range of 0.5 to 50 microns.

Additional embodiment according to the present invention is that the formulated product is a stabilized suspension.

In yet another embodiment of the present invention, wherein the pharmaceutical composition manufactured is suspension, which results in enhanced in-vitro dissolution profile.

Another embodiment according to the present invention, wherein the formulated product has particle size ranging from nanometer to micrometer, which results in-vitro dissolution profile about 80% to 110%.

In yet another embodiment of the present invention, the pharmaceutical composition is manufactured by number of stages including homogenization, sonication, mixing and/or evaporation by spray drying.

In another embodiment, a pharmaceutical composition as per the present invention comprises Abiraterone Acetate or pharmaceutically acceptable salt thereof.

Another embodiment of the present invention is intended to be supra-bioavailable, when compared to existing tablet dosages of 2 tablets of 500 mg and 4 tablets of 250 mg against a 1000 mg/5 mL oral liquid suspension dosage form. Hence, the therapeutic dose can be reduced according to the present invention.

In yet another embodiment of the present invention is that composition of Abiraterone Acetate comprising solubility enhancer and permeability enhancer, which are not the part of the current dosage form available in market.

Another embodiment of the present invention is the liquid formulation administered orally once a day, while existing dosage form is administered as four Abiraterone tablets. Tables are not suitable for old age patients because they cannot take it easily and some drugs cause gastric irritation when they are given in the tablet form. Further, tablet is not suitable for unconscious patients. Moreover, cancer patients are usually on a numerous drug regimen demanding the administration of large numbers of tablets or capsules often along with intravenous therapy. Patient compliance in such a regimen can be addressed by decreasing the number of tablets or capsules administered as well as the type of dosage forms that are administered, considering the bioavailability of the administered drug. The drug bioavailability cannot be compromised to meet patient compliance.

The compositions prepared as per the present invention are subject to animal pharmacokinetic studies to establish absorption by oral route and it is observed that the extent of absorption post oral administration ranges from 5% to 90%. Moreover, the pharmaceutical composition manufactured as per the present invention is a suspension.

Suspension eases the delivery of low soluble therapeutic agents. Therapeutic agents which are lowly soluble can be administered through a suspension. However, they require a large volume of a solvent. This volume may compromise the storage of the agent when it comes to precipitation issues.

Furthermore, suspension facilitates the dispersion of bitter drugs. Not all drugs administered to patients have a sweet taste. The majority of them are bitter especially when administered to kids who might, by all means, avoid using the drugs. Through the suspensions, the bitterness is reduced by the use of masking agents such as sweeteners and flavoring agents so that the final formulation is palatable. It is also an alternative method of administering drugs to patients who have difficulties swallowing solid drugs.

Moreover, suspensions also increase the bioavailability of drug dosage. Most drug dosages might appear in just one form such as capsule only. This form might restrict the way of administering the drug to specific patients. Through the suspension form, this challenge can be solved.

In yet another embodiment of the present invention, the pH of the pharmaceutical composition is in the range of 3.5-6.5.

In one embodiment, process for preparation of an oral liquid pharmaceutical composition as per the present invention comprising following steps:
(a) adding diluent vehicle in purified water;
(b) adding co-solvent and homogenization;
(c) adding solubilizer and homogenization;
(d) adding Abiraterone Acetate and homogenization to get homogeneous suspension;
(e) adding sweetening agent in above-prepared suspension and mixing the same through vortex or high-speed homogenizer or mechanical or sonicated or shear stress to obtain a uniform suspended formulated dosage form;
(f) adding flavouring agent and homogenization;
(g) dissolving suspending agent and thickening agent in purified water;
(h) adding solution of Step (g) into the Step (h) to provide appropriate viscosity, making-up volume of the suspension with aqueous vehicle.

The invention will be further described with respect to the following example. However, the scope of the invention is not limited thereby. All percentages stated in this specification are by weight, unless otherwise specified. While the present invention has been described in terms of its specific embodiments, certain modifications and equivalents will be apparent to those skilled in the art and are intended to be included within the scope of the present invention.

### Example 1

TABLE 1
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1296.00 | 25.92 |
| 3 | Tween 80 | 25.00 | 0.50 |
| 4 | Polyethylene glycol 400 | 100.00 | 2.00 |
| 5 | Sucralose | 2.00 | 0.04 |
| 6 | Hydroxypropyl methyl | 75.00 | 1.50 |
| | cellulose (HPMC K4M) | | |
| 7 | Strawberry flavor | 2.00 | 0.04 |
| 8 | Purified water | Q.S to make 5 mL | 50.00 |
| | | (2500.00 mg eq. | |
| | | to 2.510 mL) | |

| | | |
|---|---|---|
| Total Weight | 5000.00 | 100.00 |

TABLE 2
Result

| Sr. No. | Parameter | Result(s) |
|---|---|---|
| 1 | Assay | 99.5% |
| 2 | Dissolution (at 10 minutes) | 98.5% |

TABLE 3
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1296.00 | 25.92 |
| 3 | Tween 80 | 25.00 | 0.50 |
| 4 | Polyethylene glycol 400 | 100.00 | 2.00 |
| 5 | Sucralose | 2.00 | 0.04 |
| 6 | Hydroxypropyl cellulose | 75.00 | 1.50 |
| | (HPC-M) | | |
| 7 | Strawberry flavor | 2.00 | 0.04 |
| 8 | Purified water | Q.S to make 5 mL | 50.00 |
| | | (2500.00 mg eq. | |
| | | to 2.510 mL) | |

| | | |
|---|---|---|
| Total Weight | 5000.00 | 100.00 |

### Example 2

TABLE 4
Result

| Sr. No. | Parameter | Result(s) |
|---|---|---|
| 1 | Assay | 101.1% |
| 2 | Dissolution (at 10 minutes) | 100.4% |

### Example 3

TABLE 5
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1358.50 | 27.17 |
| 3 | Tween 80 | 25.00 | 0.50 |
| 4 | Polyethylene glycol 400 | 100.00 | 2.00 |
| 5 | Sucralose | 2.00 | 0.04 |
| 6 | Povidone K120 | 250.00 | 5.00 |
| 7 | Strawberry flavor | 2.00 | 0.04 |
| 8 | Purified water | Q.S to make 5 mL | 45.25 |
| | | (2262.5 mg eq. | |
| | | to 2.272 mL) | |

| | | |
|---|---|---|
| Total Weight | 5000.00 | 100.00 |

TABLE 6
Result

| Sr. No. | Parameter | Result(s) |
|---|---|---|
| 1 | Assay | 98.7% |
| 2 | Dissolution (at 10 minutes) | 97.2% |

### Example 4

TABLE 7
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1394.00 | 27.88 |
| 3 | Tween 80 | 25.00 | 0.50 |
| 4 | Polyethylene glycol 400 | 100.00 | 2.00 |
| 5 | Sucralose | 2.00 | 0.04 |
| 6 | Colloidal silicone dioxide | 50.00 | 1.00 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 8
Result

| Sr. No. | Parameter | Result(s) |
|---|---|---|
| 1 | Assay | 104.12% |
| 2 | Dissolution (at 10 minutes) | 99.60% |

### Example 5

TABLE 9
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Tween 80 | 25.00 | 0.50 |
| 4 | Polyethylene glycol 400 | 100.00 | 2.00 |
| 5 | Sucralose | 2.00 | 0.04 |
| 6 | Xanthan gum | 12.50 | 0.25 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 10
Result

| Sr. No. | Parameter | Result(s) |
|---|---|---|
| 1 | Assay | 104.12% |
| 2 | Dissolution (at 10 minutes) | 99.60% |

### Example 6

TABLE 11
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Soluplus | 25.00 | 0.50 |
| 4 | Polyethylene glycol 400 | 100.00 | 2.00 |
| 5 | Sucralose | 2.00 | 0.04 |
| 6 | Xanthan gum | 12.50 | 0.25 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 12
Result

| Sr. No. | Parameter | Result(s) |
|---|---|---|
| 1 | Assay | 101.4% |
| 2 | Dissolution (at 10 minutes) | 95.6% |

### Example 7

TABLE 13
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Polyoxyl 40 Hydrogenated | 25.00 | 0.50 |
| | Castor Oil (Kolliphor ® RH 40) | | |
| 4 | Polyethylene glycol 400 | 100.00 | 2.00 |
| 5 | Sucralose | 2.00 | 0.04 |
| 6 | Xanthan gum | 12.50 | 0.25 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 14
Result

| Sr. No. | Parameter | Result(s) |
|---|---|---|
| 1 | Assay | 102.6% |
| 2 | Dissolution (at 10 minutes) | 96.4% |

### Example 8

TABLE 15
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Caprylocaproyl Polyoxyl-8 | 25.00 | 0.50 |
| | glycerides (Labrasol ALF) | | |
| 4 | Polyethylene glycol 400 | 100.00 | 2.00 |
| 5 | Sucralose | 2.00 | 0.04 |
| 6 | Xanthan gum | 12.50 | 0.25 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 16
Result

| Sr. No. | Parameter | Result(s) |
|---|---|---|
| 1 | Assay | 102.1% |
| 2 | Dissolution (at 10 minutes) | 103.5% |

### Example 9

TABLE 17
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Lauroyl polyoxyl-6 glycerides | 25.00 | 0.50 |
| 4 | Polyethylene glycol 400 | 100.00 | 2.00 |
| 5 | Sucralose | 2.00 | 0.04 |
| 6 | Xanthan gum | 12.50 | 0.25 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 18
Result

| Sr. No. | Parameter | Result(s) |
|---|---|---|
| 1 | Assay | 100.9% |
| 2 | Dissolution (at 10 minutes) | 100.4% |

### Example 10

TABLE 19
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Lauroyl Polyoxyl-32 | 25.00 | 0.50 |
| | glycerides (Gelucire 44/14) | | |
| 4 | Polyethylene glycol 400 | 100.00 | 2.00 |
| 5 | Sucralose | 2.00 | 0.04 |
| 6 | Xanthan gum | 12.50 | 0.25 |
| 7 | Strawberry flavor | 4.00 | 0.08 |
| 8 | Purified water | Q.S to make 5 mL | 48.50 |
| | | (2425 mg eq. | |
| | | to 2.435 mL) | |

| | | |
|---|---|---|
| Total Weight | 5000.00 | 100.00 |

TABLE 20
Result

| Sr. No. | Parameter | Result(s) |
|---|---|---|
| 1 | Assay | 97.9% |
| 2 | Dissolution (at 10 minutes) | 95.6% |

### Example 11

TABLE 21
Formulation

| | | Quantity/Dose (mg) | |
|---|---|---|---|
| | | Strength: | |
| Sr. No. | Ingredient(s) | 1000 mg/5 mL | % W/V |
| 1 | Abiraterone Acetate | 1000.00 | 20.00 |
| 2 | Sorbitol solution 70% | 1431.50 | 28.63 |
| 3 | Tween 80 | 25.00 | 0.50 |
| 4 | Polyethylene glycol 400 | 100.00 | 2.00 |
| 5 | Sucralose | 4.00 | 0.08 |
| 6 | Xanthan gum | 12.50 | 0.25 |
| 7 | Strawberry flavor | 4.00 | 0.08 |
| 8 | Purified water | Q.S to make 5 mL | 48.46 |
| | | (2423 mg eq. | |
| | | to 2.423 mL) | |

| | | |
|---|---|---|
| Total Weight | 5000.00 | 100.00 |

TABLE 22
Result

| Sr. No. | Parameter | Result(s) |
|---|---|---|
| 1 | Assay | 100.8% |
| 2 | Dissolution (at 10 minutes) | 99.10% |

### Common Manufacturing Process for Example 1-11

1. Take purified water (half quantity) in container.
2. Add Sorbitol into the Step 1 under stirring & mix it for 10 minutes with help of homogenizer.
3. Add Polyethylene glycol 400 into solution of Step 2 under homogenization and continue the homogenization for 10 minutes.
4. Add Tween 80/Soluplus/Polyoxyl 40 Hydrogenated Castor Oil (Kolliphor® RH 40)/Caprylocaproyl Polyoxyl-8 glycerides (Labrasol ALF)/Lauroyl polyoxyl-6 glycerides (Labrafil M 2130 CS)/Lauroyl Polyoxyl-32 glycerides (Gelucire 44/14) into the solution of Step 3 under homogenization and continue the homogenization for 10 minutes.
5. Add Abiraterone Acetate API into the mixture of Step 4 under homogenization and continue the homogenization for 1 hour to get uniform homogeneous suspension.
6. Add Sucralose into the suspension of Step 5 under homogenization and continue the homogenization for 10 minutes.
7. Add Strawberry flavor into the suspension of Step 6 under homogenization and continue the homogenization for 10 minutes.
8. Dissolve Hydroxypropyl methyl cellulose (HPMC K4M)/Hydroxypropyl cellulose (HPC-M)/Povidone K120/Colloidal silicone dioxide/Xanthan gum in purified water under homogenization and continue the homogenization for 30 minutes.
9. Add solution of Step 8 into the Step 7 under homogenization to make sufficient volume up and continue the homogenization for 60 minutes.
10. Perform wet milling of above suspension through DYNO®-MILL with desired parameters.
11. Collect wet milled suspension in containers.

### Stage: Wet Milling Through Dyno Mill:

Dissolution of Abiraterone Acetate
Oral Liquid Suspension 1000 mg/5 mL

| Dissolution Media: | 0.25% sodium lauryl sulfate in | |
|---|---|---|
| | 56.5 mM phosphate buffer | |
| Dissolution Media Volume | 900 | ml |
| pH | 5.15 | |
| USP Apparatus | Paddle | |
| RPM | 50 | RPM |

% Drug Release of Abiraterone Acetate

| Time point (min) | Initial | Dyno Mill |
|---|---|---|
| 5 | 95.50 | 97.6 |
| 10 | 97.00 | 98.9 |
| 15 | 97.10 | 99.1 |
| 20 | 98.70 | 99.4 |
| 30 | 99.20 | 99.2 |
| 45 | 97.70 | 99.8 |
| Assay (%) | 102.0 | 100.8 |

### Accelerated Stability Report:

Assay, pH and dissolution profile studies were incorporated at various conditions and time periods to evaluate the stability up to 6 months.

The dissolution profile studies were observed in media as defined by USFDA, which is having 50 rpm in 900 mL of pH 4.5 buffer with 0.25% sodium lauryl sulfate in 56.5 mM phosphate buffer.

For this stability study, the oral liquid suspension composition prepared according to Example-11 were observed.

### Test Example 1

All the samples were charged for stability study at accelerated condition at 40±2° C. and 75±5% RH and chemical parameters were evaluated at initial, after 1 month, after 3 months and after 6 months. The results obtained are reported in the table below:

TABLE 23
Accelerated Stability Conditions

| | |
|---|---|
| Temperature Range | 40 ± 2° C. |
| % Relative Humidity (% RH) | 75 ± 5% RH |
| Time Interval | initial, after 1 month, after |
| | 3 months and after 6 months |

Results

| Time Interval | Assay (%) | pH | Dissolution |
|---|---|---|---|
| Initial | 104.12 | 5.70 | 99.6 |
| 1 month | 104.33% | 4.85 | 104.00 |
| 3 month | 100.18% | 6.29 | 101.30 |
| 6 month | 98.76% | 6.31 | 99.90 |

### Test Example 2

All the samples were charged for stability study at accelerated condition at 30±2° C. and 75±5% RH and chemical parameters were evaluated at initial, after 1 month, after 3 months and after 6 months. The results obtained are reported in the table below:

TABLE 24
Accelerated Stability Conditions

| | |
|---|---|
| Temperature Range | 30 ± 2° C. |
| % Relative Humidity (% RH) | 75 ± 5% RH |
| Time Interval | initial, after 1 month, after |
| | 3 months and after 6 months |

Results

| Time Interval | Assay (%) | pH | Dissolution |
|---|---|---|---|
| Initial | 104.12 | 5.70 | 99.6 |
| 1 month | 100.66% | 5.01 | 100.66% |
| 3 month | 99.06% | 6.17 | 99.06% |
| 6 month | 99.91% | 6.26 | 99.91% |

### Test Example 3

All the samples were charged for stability study at accelerated condition at 25±2° C. and 60±5% RH and chemical parameters were evaluated at initial, after 1 month, after 3 months and after 6 months. The results obtained are reported in the table below:

TABLE 25
Accelerated Stability Conditions

| | |
|---|---|
| Temperature Range | 25 ± 2° C. |
| % Relative Humidity (% RH) | 60 ± 5% RH |
| Time Interval | initial, after 1 month, after |
| | 3 months and after 6 months |

Results

| Time Interval | Assay (%) | pH | Dissolution |
|---|---|---|---|
| Initial | 104.12 | 5.70 | 99.6 |
| 1 month | 100.85% | 5.49 | 95.5 |
| 3 month | 100.04% | 6.13 | 99.1 |
| 6 month | 100.98% | 6.19 | 98.5 |

As per the above test examples, there is clear indication that the Assay, pH and dissolution profile studies of the composition as per the present invention is stable up to 6-months. As such, there is no need to add pH adjusting agent for pH adjustment in the final formulation. Therefore, above results demonstrate that the present novel invention surprisingly produces better results and proves its inventiveness by technical advancement.

### Comparative Study Report

Assay and dissolution profile studies were evaluated and it was established that the composition prepared as per the present invention against marketed formulation (Zytiga® 250 mg & 500 mg) is comparable. The dissolution profile studies were observed in media as defined by USFDA.

TABLE 26
Comparison of % Assay for Abiraterone
Acetate 500 mg formulations

| ZYTIGA ® 500 mg Tablets | Oral Liquid Suspension of |
|---|---|
| Abiraterone Acetate | Abiraterone Acetate |
| 97.10% | 98.60% |

TABLE 27
Comparison of Dissolution Profile for
Abiraterone Acetate 500 mg formulations

| Dissolution Media | 0.25% sodium lauryl sulfate in | |
|---|---|---|
| | 56.5 mM phosphate buffer | |
| Dissolution Media Volume | 900 | ml |
| pH | 4.5 | |
| USP Apparatus | Paddle | |
| RPM | 50 | RPM |

% Drug Release of Abiraterone Acetate

| | | ZYTIGA ® 500 mg | |
|---|---|---|---|
| Time Point | ZYTIGA ® 500 mg | Two Tablets | Oral Liquid |
| (min) | One Tablet | 1000 mg Strength | Suspension |
| 05 | — | 28.60 | 95.50 |
| 10 | 62.3 | 54.00 | 97.00 |
| 15 | 84.3 | 70.70 | 97.10 |
| 20 | 94.1 | 79.10 | 98.70 |
| 30 | 96.9 | 84.10 | 99.20 |
| 45 | 96.5 | 86.20 | 97.70 |

As per the above comparison, there is clear indication that the dissolution of the suspension 1000 mg/5 mL against the Zytiga® 1000 mg (by using 500 mg×2 tablets), incomplete release is observed in the innovator product indicating that the suspension has good solubility in existing media and rate of solubilisation is more as compared to the initial to the extended time period. Considering the dissolution profile, it is concluded that suspension formulation has high rate and the extent of absorption when administered in in-vivo.

The invention described herein comprises in various objects as mentioned above and their description in relation to characteristics, compositions and process adopted. While these aspects are emphasised in the invention, any variations of the invention described above are not to be regarded as departure from the spirit and scope of the invention as described.

The above-mentioned examples are provided for illustrative purpose only and these examples are in no way limitative on the present invention.

1. A pharmaceutical liquid suspension formulation suitable for oral administration comprising abiraterone acetate or a pharmaceutically acceptable salt thereof and one or more pharmaceutically acceptable excipients.

2. The pharmaceutical liquid suspension formulation as claimed in claim 1, comprising abiraterone acetate or a pharmaceutically acceptable salt thereof in the form of solid particles having a particle size distribution wherein D90 is in the range of 0.5 to 50 microns.

3. The pharmaceutical liquid suspension formulation as claimed in claim 1, comprising about 5% to 60% w/v of abiraterone acetate and the one or more pharmaceutically acceptable excipients.

4. The pharmaceutical liquid suspension formulation as claimed in claim 1, wherein the one or more pharmaceutically acceptable excipients comprise diluent vehicles, stabilizers/anti-oxidants, suspending/thickening agents, chelating/complexing agents, solubility enhancing agents, permeability enhancers, preservatives, glidants, active carriers, sweeteners, anti-caking agents, wetting agents, preservatives, buffering agents, flavoring agents, or a mixture thereof.

5. The pharmaceutical liquid suspension formulation as claimed in claim 1, comprising
(a) 5% to 60% w/v of abiraterone acetate;
(b) 15% to 70% w/v of diluent vehicles,
(c) 0.05% to 5% w/c of solubilizers,
(d) 0.1% to 10% w/v of co-solvent;
(e) 0.001% to 1% w/v of sweeteners,
(f) 0.025% to 2.5% w/v of thickening agents, and
(g) 0.001% to 1% w/v of flavoring agent.

6. The pharmaceutical liquid suspension formulations as claimed in claim 1, comprising:
(a) abiraterone acetate, in a concentration from about 5% by weight to about 60% by weight of the pharmaceutical liquid suspension formulation;
(b) a diluent vehicle component in a concentration from about 15% by weight to about 70% by weight of the pharmaceutical liquid suspension formulation, said diluent vehicle component comprising at least one diluent vehicle component selected from the group consisting of aqueous vehicle, sugar, methylcellulose gel, citric acid, sucrose, sorbitol solution, sodium carboxy methylcellulose solution, xanthan gum solution, non-aqueous vehicle like refined fractionated coconut oil, hydrogenated castor oil, lecithin, and aluminum stearate;
(c) a solubilizer component in a concentration from about 0.05% by weight to about by weight of the pharmaceutical liquid suspension formulation, said solubilizer component comprising at least one solubilizer selected from the group consisting of Tween 80, Soluplus, Polyoxyl 40 Hydrogenated Castor Oil (Kolliphor® RH 40), Caprylocaproyl Polyoxyl-8 glycerides (Labrasol ALF), Lauroyl polyoxyl-6 glycerides (Labrafil M 2130 CS), Lauroyl Polyoxyl-32 glycerides (Gelucire 44/14), Cremophor, and Lecithin;
(d) a co-solvent component in a concentration from about 0.1% by weight to about 10 by weight of the pharmaceutical liquid suspension formulation, said co-solvent component comprising at least one co-solvent selected from the group consisting of ethanol, propylene glycol, glycerine, glycofural, and polyethylene glycols;
(e) a sweetener component in a concentration from about 0.001% by weight to about 1% by weight of the pharmaceutical liquid suspension formulation, said sweetener component comprising at least one pharmaceutically acceptable sweetener;
(f) a thickening agent component in a concentration from about 0.025% by weight to about 2.5% by weight of the pharmaceutical liquid suspension formulation, said thickening agent component comprising at least one thickening agent selected from the group consisting of sodium alginate, methylcellulose, hydroxyethyl cellulose, hydroxypropyl cellulose, hydroxypropyl methylcellulose, povidone K 120, colloidal silicon dioxide, carboxymethyl cellulose, sodium carboxymethyl cellulose, acacia, tragacanth, xanthan gum, carbomer, carrageen, and gelatin; and
(g) a flavoring component in a concentration from about 0.001% by weight to about 1% by weight of the pharmaceutical liquid suspension formulation, said flavoring component comprising at least one pharmaceutically acceptable flavoring agent.

7. The a pharmaceutical liquid suspension formulation as claimed in claim 1, manufactured by following steps:
(a) adding sorbitol solution in purified water;
(b) adding polyethylene glycol and homogenization;
(c) adding tween 80 and homogenization;
(d) adding abiraterone acetate and homogenization to get homogeneous suspension;
(e) adding sweetening agent in above-prepared suspension and mixing the same through vortex or high-speed homogenizer or mechanical or sonicated or shear stress to obtain a uniform suspended formulated dosage form;
(f) adding flavouring agent and homogenization;
(g) dissolving xanthan gum in purified water; and
(h) adding solution of Step (g) into the Step (h) to provide appropriate viscosity, making-up volume of the suspension with purified water.

8. The pharmaceutical liquid suspension formulation as claimed in claim 1, wherein a pH of the pharmaceutical liquid suspension formulation is in the range of 3.5-6.5.
